(19) United States
(12) Patent Application Publication
(10) Pub. No.: US 2003/0054430 A1
(43) Pub. Date: Mar. 20, 2003

(54) Stoichiometry measurements for the parameterization of absolute rate models for cytochrome P450 metabolism

(75) Inventors: Kenneth R. Korzekwa, Mountain View, CA (US); Jeffrey P. Jones, Pullman, WA (US); Timothy J. Carlson, Mountain View, CA (US); Jan L. Wahlstrom, Fremont, CA (US); Marc J. Evanchik, Fremont, CA (US)

Correspondence Address: BEYER WEAVER & THOMAS LLP, P.O. BOX 778, BERKELEY, CA, 94704-0778, US

(73) Assignee: Camitro Corporation

(21) Appl. No.: 09/954,726

(22) Filed: Sep. 12, 2001

(51) Int. Cl. G06G 7/48; G06G 7/58; G06F 19/00; G01N 33/48; G01N 33/50; C12Q 1/26

(52) U.S. Cl. 435/25; 702/19; 703/11

(57) ABSTRACT

Systems and method are provided for modeling substrate molecules so that the various pathway reaction rates, and thus their overall reaction rates and metabolic properties, can be predicted. The current invention provides various systems and methods for stoichiometrically measuring the pathway reaction rates, both directly and indirectly. By repeating this for a class or several classes of substrate molecules, a general model of pathway reaction rates can be developed by correlating observed pathway reaction rates to the actual structural descriptors of the molecules, in particular, features around the reactive sites. The model can then be used to predict and design substrates according to desired metabolic characteristics. The systems and methods are particularly applicable to metabolism of substrate molecules by the cytochrome P450 enzymes.

### CROSS-REFERENCE TO RELATED APPLICATIONS

This patent application is related to U.S. patent application Ser. No. 09/368,511, “Use of Computational and Experimental Data to Model Organic Compound Reactivity in Cytochrome P450 Mediated Reactions and to Optimize the Design of Pharmaceuticals,” filed Aug. 5, 1998 by Korzekwa et al.; U.S. patent application Ser. No. 09/613,875, “Relative Rates of Cytochrome P450 Metabolism,” filed Jul. 10, 2000, by Korzekwa et al.; U.S. Provisional Patent Application Patent Application No. 60/217,227, “Accessibility Correction Factors for Quantum Mechanical and Molecular Models of Cytochrome P450 Metabolism,” filed Jul. 10, 2000; and U.S. patent application Ser. No. 09/902,470, “Accessibility Correction Factors For Electronic Models Of Cytochrome P450 Metabolism,” filed Jul. 9, 2001 by Korzekwa et al.; and U.S. patent application Ser. No. 09/811,283, “Predicting Metabolic Stability Of Drug Molecules,” filed Mar. 15, 2001 by Korzekwa et al. These patent applications, as well as any other patents, patent application and publications cited herein, are hereby incorporated by reference in their entirety for all purposes.

The U.S. Government may have certain rights in this invention pursuant to National Institute of Environmental Health Sciences Grant No. 1RO1ES09122.

### FIELD OF THE INVENTION

The present invention relates generally to systems and methods for modeling the reaction pathways of substrate molecules, especially drugs. More specifically, the invention relates to systems and methods for modeling the various reaction pathways associated with substrate molecules residing in reactive sites of the cytochrome P450 enzymes. Further, the invention relates to systems and methods for conducting direct and indirect stoichiometry measurements of such reaction pathways in order to model, predict and design the metabolic properties of such substrate molecules.

### BACKGROUND OF THE INVENTION

Drug development is an extremely expensive and lengthy process. The cost of bringing a single drug to market is about $500 million to $1 billion dollars, with the development time being about 8 to 15 years. Drug development typically involves the identification of 1000 to 100,000 candidate compounds distributed across several compound classes that eventually lead to a single or at most a few marketable drugs.

Those thousands of candidate compounds are screened against biochemical targets to assess whether they have the pharmacological properties that the researchers are seeking. This screening process leads to a much smaller number of “hits” (perhaps 500 or 1000) which bind with a target receptor and which are narrowed to even fewer “leads” (perhaps 50 or 100) which appear most efficacious. At this point, typically, the lead compounds are assayed for their ADME/PK (absorption, distribution, metabolism, and elimination/pharmokinetic) properties. They are tested using biochemical assays such as Human Serum Albumin binding, chemical assays such as pK_{A} and solubility testing, and in vitro biological assays such as metabolism by endoplasmic reticulum fractions of human liver, in order to estimate their actual in vivo ADME/PK properties. Most of the lead compounds are discarded because of unacceptable ADME/PK properties.

In addition, even optimized leads that have passed these tests and are submitted for FDA clinical trials as investigational new drugs (INDs) will often show undesirable ADME/PK properties when actually tested in animals and humans. Abandonment or redesign of optimized leads at this stage is extremely costly, since FDA trials require formulation, manufacturing and extensive testing of the compounds.

The development of compounds with unacceptable ADME/PK properties thus contributes greatly to the overall cost of drug development. If there was a process by which compounds could be discarded or redesigned at an earlier stage of development (the earlier the better), then great savings in terms of money and time could be achieved. The current tools essentially offer no comprehensive method by which this can be done.

A large portion of all drug metabolism in humans and most all higher organisms is carried out by the cytochrome P450 enzymes. The cytochrome P450 enzymes (CYP) are a superfamily of heme-containing enzymes that include more than 700 individual isozymes that exist in plant, bacterial and animal species. Nelson et al., Pharmacogenetics. 6, 1-42 (1996). They are monooxygenase enzymes. Wislocki et al., in Enzymatic Basis of Detoxification (Jakoby, Ed.), 135-83 (1980), Academic Press, New York. Although humans share the same several CYP enzymes, these enzymes can vary slightly between individuals (alleles) and the enzyme profile of individuals, in terms of the amount of each enzyme that is present, also varies to some degree.

It is estimated that in humans, 50% of all drugs are metabolized partly by the P450 enzymes, and 30% of drugs are metabolized primarily by these enzymes. The most important CYP enzymes in drug metabolism are the CYP3A4, CYP2D6 and CYP2C9 isozymes. While modeling techniques do exist for predicting substrate metabolism by enzymes other than CYP enzymes, no sufficiently accurate technique exists for modeling metabolism by the CYP enzymes. To the extent that modeling techniques are available for other enzymes, they work by analyzing either the interactions between enzyme and substrate, or the common characteristics for a series of substrates. See, for example, Schramrn et al., Annu Rev Biochem, 67: 693-720 (1998); Hunter et al., Parasitology, 114 Suppl: S17-29 (1997); Gschwend et al, Mol Recognit, Mar-Apr; 9(2): 175-86 (1996).

While these modeling techniques are partially effective for some enzymes, they are frequently ineffective for the CYP enzymes. This is because the CYP enzymes lack the high binding specificities that characterize most other enzymes. CYP3A is almost completely nonspecific from a binding perspective, while CYP2D6 and CYP2C9 are only modestly specific. Gross steric and electrostatic properties of a substrate have, at most, a secondary effect on their metabolism by the CYP enzymes. Thus modeling techniques in the current art cannot be used to model CYP enzyme metabolism.

In view of the importance of the CYP enzymes to drug metabolism, a modeling technique for CYP-substrate interaction and metabolism would be highly beneficial. Such a technique would provide researchers with valuable ADME/PK information on compounds at an early stage in the development process.

### SUMMARY OF THE INVENTION

The present invention addresses this need by providing systems and methods for modeling substrate molecules so that their pathway reaction rates, and thus overall metabolic properties, can be modeled and predicted. The invention provides various techniques for stoichiometrically measuring the reaction pathways of a substrate molecule as it is catalyzed by a CYP enzyme, either by directly or indirectly measuring the reactants and/or products. Once these measurements are made on a class of substrate molecules or on several classes, the propensity of the molecule for reaction along a particularly pathway can be modeled and predicted based on the molecule's structure, particularly the structural features near the reactive sites of the molecules.

In one embodiment of the invention, the reaction rates of different pathways in the CYP catalytic cycle are determined for a substrate molecule. Three reaction vessels (e.g., cuvettes or microtiter wells) are used to make the necessary measurement. In the first reaction vessel, an oxygen electrode or a ruthenium complex matrix (or other species or tool for directly detecting oxygen) is used to determine the concentration of oxygen. The second vessel contains at least the substrate molecule, a CYP enzyme, and NADPH. The third vessel contains at least the substrate molecule, the CYP enzyme, NADPH, as before, and the enzyme catalase. The NADPH consumption in each of these vessels is measuring using UV absorption techniques. The formation of product (oxidized substrate) is determined through chromatography or mass spectrometry or another technique well known in the art.

From these three measurements, the absolute reaction rate for all the relevant pathways involving the substrate and the enzyme are determined. The process is then typically repeated for a class of substrates or several classes so that a model of substrate metabolism can be constructed. This model correlates the reaction rates of the different pathways with the molecular structure of a substrate molecule, particularly in the vicinity of the molecule's reactive sites. The invention described in U.S. patent application Ser. No. 09/613,875 (Atty Docket No.: CAMIP002) can be practiced with the current invention to yield more precise models of substrate metabolism, particularly with respect to the several sites of metabolism (reactive sites) that a substrate molecule may have.

One aspect of the invention pertains to methods for calculating the rate or amount of water decoupling for a substrate molecule in the CYP catalytic cycle. The method includes measuring oxygen and NADPH consumption and product formation. The method includes calculating a reaction rate for the water-decoupling pathway based on the difference between oxygen and NADPH consumption. The method can be repeated for one or more substrate molecules so that a generalized model of substrate metabolism can be generated. The oxygen consumption can be measured using an oxygen electrode or a ruthenium complex matrix, for example.

Another aspect of the invention pertains to methods for calculating the rate or amount of peroxide decoupling for a substrate molecule in the CYP catalytic cycle. The method includes measuring oxygen and NADPH consumption and product formation. The method also includes adding catalase and measuring the amount of oxygen generated after addition of the catalase. The method can be repeated for one or more substrate molecules so that a generalized model of substrate metabolism can be generated. The oxygen consumption is calculated from the amount of oxygen generated after addition of the catalase. Again, the oxygen consumption can be measured using an oxygen electrode or a ruthenium complex matrix.

Yet another aspect of the invention pertains to methods for modeling reaction rates of substrate molecules in cytochrome P450 metabolism. The method includes receiving or generating a molecular structure for a substrate, a substrate oxidation rate, and a hydrogen peroxide decoupling reaction rate. Typically, the method will also receive or generate a water-decoupling rate. From this information, the method predicts a metabolism rate for the substrate. Importantly, the process receives or predicts a peroxide-decoupling rate for the substrate in the cytochrome P450 enzyme. Another aspect of the invention provides for similar methods for modeling reaction rates by creating and using computational models that account for the peroxide-decoupling rate, the water decoupling rate and the product formation rate of a given substrate.

Another aspect of the invention pertains to methods for predicting the relative reaction velocities of a first and second reaction pathway of a substrate molecule in cytochrome P450 metabolism, including analyzing the molecular structure of the molecule to see if is has a particular structural feature and predicting whether the first reaction pathway is preferred. The first reaction pathway is typically oxene formation (on the way to ultimately producing product), and the second reaction pathway is typically the peroxide decoupling pathway. A prediction can be made based on whether the structural feature does or does not exclude water from the reactive site, or whether the structural feature is hydrophobic or hydrophilic.

Another aspect of the invention pertains to methods for analyzing a substrate molecule and its first reaction pathway that forms a product and a second reaction pathway that forms water and regenerates the substrate by determining the change in concentration in NAPDH and oxygen during a reaction of the substrate and thereby estimating the relative value contribution of the second pathway. The estimation is typically based on the difference in the change in concentration of NADPH and oxygen. The method can be used for analysis of substrate metabolism by cytochrome P450.

Yet another aspect of the invention pertains to computer program products including machine-readable media on which are provided program instructions for implementing the methods described above, in whole or in part. Many of the methods of this invention may be represented, in whole or in part, as program instructions that can be provided on such machine-readable media. In addition, the invention pertains to various combinations and arrangements of data generated and/or used as described herein.

These and other features of the present invention will be described in more detail below in the detailed description of the invention and in conjunction with the following figures.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a schematic illustration of the mammalian cyctochrome P450 catalytic cycle, including the non-metabolic decoupling reactions.

FIG. 2 is a schematic illustration of a substrate molecule (drug) with several reactive sites.

FIG. 3A is a schematic illustration of the active site of a CYP enzyme, specifically P450 CAM.

FIG. 3B is a schematic illustration of the heme iron atom of P450 CAM coordinated with molecular oxygen.

FIGS. 3C and 3D present a process flow diagram for a software tool used to predict the reaction rates of the reaction sites on a substrate molecule.

FIG. 3E shows how the process of FIGS. 3C and 3D might treat an anisole molecule, which has both an aliphatic and aromatic reactive sites.

FIG. 4A presents the chemical equations for the product formation, hydrogen peroxide decoupling and water decoupling pathways in CYP metabolism.

FIG. 4B summarizes the stoichiometric coefficients for the chemical equations of FIG. 4A in table form.

FIG. 5A presents a curve of NADPH concentration versus time in an NADPH limited reaction system.

FIG. 5B presents a curve of oxygen concentration versus time for reaction system that generated the curve of FIG. 5A. It also shows the effect of adding catalase when hydrogen peroxide has been generated in the reaction system.

FIG. 5C presents overlaid curves of NADPH and oxygen concentration generated for a reaction system in which the water decoupling reaction occurs—as indicated by the different slopes in the NADPH and oxygen curves.

FIG. 6A presents a curve of NADPH versus time for an oxygen limited system.

FIG. 6B presents a curve of oxygen versus time for the oxygen limited system of FIG. 6A.

FIG. 6C presents a curve as in FIG. 6A, but with catalase added when the hydrogen peroxide decoupling reaction occurs.

FIG. 6D presents a curve of oxygen versus time for the reaction system of FIG. 6C.

FIG. 7A schematically illustrates a well with a cap that is designed to create a steep meniscus to limit the diffusion of oxygen into the reaction solution.

FIG. 7B schematically illustrates a reaction vessel with a ruthenium-complex polyacrylamide matrix and a fiber optic probe.

FIG. 7C schematically illustrates a reaction vessel including a light source and a detector for measuring optical absorbance of a reaction solution, such as an NADPH solution.

FIG. 8 schematically illustrates a set of three microtitre wells that is used in a preferred embodiment to determine oxygen and NADPH consumption and water and hydrogen peroxide formation.

FIGS. 9A and 9B illustrate a computer system suitable for implementing embodiments of the present invention.

FIG. 10 is a block diagram of an Internet based system for predicting metabolic properties of molecules in accordance with an embodiment of the present invention.

### DETAILED DESCRIPTION

In the following detailed description of the present invention, numerous specific embodiments are set forth in order to provide a thorough understanding of the invention. However, as will be apparent to those skilled in the art, the present invention may be practiced without these specific details or by using alternate elements or processes. In other instances well known processes, procedures and components have not been described in detail so as not to unnecessarily obscure aspects of the present invention.

1. INTRODUCTION

A “metabolic enzyme” as used herein refers to any enzyme that is involved in xenobiotic metabolism. Many metabolic enzymes are involved in the metabolism of exogenous compounds. Metabolic enzymes include enzymes that metabolize drugs, such as the CYP enzymes, uridine-diphosphate glucuronic acid glucuronyl transferases and glutathione transferases.

“Xenobiotic metabolism” as used herein refers to any and all metabolism of foreign molecules that occurs in living organisms, including anabolic and catabolic metabolism.

A “reactive site” as used herein refers to a site on a substrate molecule that is susceptible to metabolism and/or catalysis by an enzyme. It is to be distinguished from an “active site,” which is the region of an enzyme that is involved in catalysis.

A “main pathway” as used herein refers to any chemical reaction pathway of particular interest. A main pathway will have a “branch pathway” that is an alternate reaction to the main pathway. The branch pathway typically yields a different product or products than the main pathway. In metabolic enzymes, branch pathways may provide “decoupling reactions” which produce non-metabolic products. In such enzymes, the main pathway is the reaction pathway to substrate metabolism. The CYP catalytic cycle, which will be discussed in more detail below, is believed to have three decoupling reactions, one decoupling to superoxide, one decoupling to hydrogen peroxide, and one decoupling to water. Each of these branch pathways produces the original, unmodified substrate. Thus, when any of these branch pathways occur at a rate comparable to that of the main pathway, the rate of substrate metabolism slows. In some cases, the bound molecule may actually be an inhibitor of the enzyme, rather than a substrate.

A “reaction pathway” as used herein refers generically to any branched pathway or the main pathway. If a CYP metabolic enzyme is involved, then “reaction pathway” refers to any one of the four possible pathway outcomes once a molecule has complexed with the CYP enzyme. These reaction pathways are the superoxide-decoupling, peroxide-decoupling, water-decoupling and product-formation pathways. Note that the “water-decoupling pathway” is semantically distinguishable from a “water-decoupling step,” in that the latter refers only to the immediate single reaction step that decouples the water molecules (see step 112 below) while the former refers to an entire reaction cycle, starting with the molecule complexing with the enzyme and ending with the water-decoupling step in which the molecule-enzyme complex is regenerated. The other reaction pathways and reaction steps are distinguishable in a similar manner.

“Reaction rate” as used to herein refers to the kinetic rate of a chemical reaction or a single step or reaction pathway of a chemical reaction. The reaction rate can be predicted by modeling the transition state or estimating the activation energy from the difference in free energy between a substrate and an intermediate form. The term “reaction velocity” is used interchangeably with “reaction rate.”

A “complex” as used to herein is an enzyme-molecule coupling formed by covalent and/or other bonds that may or may not lead to metabolism of the molecule. If it leads to metabolism, then the molecule is a substrate. If it does not lead to metabolism, then the molecule is an inhibitor.

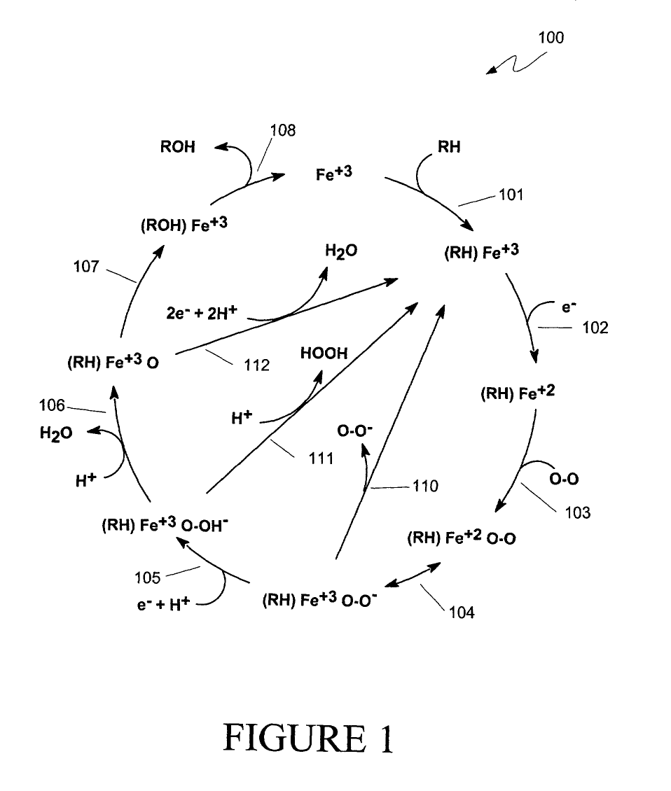

FIG. 1 illustrates the oxidative hydroxylation catalytic cycle 100 for a mammalian CYP enzyme. The top of the figure shows a generic starting substrate (RH) and generic product (ROH). This hydroxylation reaction is often the first step in metabolizing an exogenous compound, and partly explains the importance of the CYP enzymes in drug deactivation/metabolism. Note that the hydroxylated product is not the only possible oxidation product produced by CYP enzymes; it is simply presented here for the sake of illustration. In addition, the described catalytic cycle is the generally accepted mechanism, but variations may occur between different P450 enzymes.

A first step 101 of the catalytic cycle 100 shows the initial binding of the substrate RH to the heme iron atom of the enzyme, which changes the equilibrium spin state of the heme iron from low to high. This lowers the reduction potential of the iron, thus facilitating transfer of an electron from NADPH, via cytochrome P450 reductase, to the iron atom in a second step 102. In a third step 103, molecular oxygen binds to the iron atom. In a fourth step 104, the bound oxygen is reduced by one electron and the iron is oxidized from a ferrous state to a ferric state. At this point, the oxygen can be decoupled from the enzyme as superoxide in a non-metabolic reaction, thus taking the enzyme-substrate complex back to its initial state (illustrated as the product of step 101) in a branch pathway step 110. Otherwise, the oxygen combines with one more electron and a proton in a fifth step 105, forming a peroxy intermediate with the enzyme-substrate complex. Here, a hydrogen peroxide decoupling reaction can take place, as illustrated in a branch pathway step 111, which takes the enzyme-substrate complex back to the initial state (again illustrated as the product of step 101).

Otherwise, in a sixth step 106, the peroxy intermediate reacts with another proton to undergo heterolytic cleavage, with one oxygen leaving the complex as a water molecule and the other oxygen coordinating with the iron atom as a reactive oxygen atom. A water decoupling reaction involving the addition of two protons and two electrons, illustrated as a branch pathway step 112, can take the enzyme-substrate complex back to the initial state. Otherwise, the reactive oxygen is transferred to the substrate to form an oxidized product (ROH), a seventh step 107. The product ROH then dissociates from the enzyme, an eighth step 108.

Note that the superoxide decoupling reaction 110, the hydrogen peroxide decoupling reaction 111, and the water decoupling reaction 112 all yield the substrate back in its original form in complex with the enzyme. These pathways thus reduce the rate of metabolism of the substrate. If either of the decoupling pathways predominate in the CYP catalytic cycle, then the substrate is unlikely to be metabolized rapidly.

Experimental evidence for the existence of these reaction pathways and intermediates is described in U.S. patent application Ser. No. 09/368,511, by Korzekwa et al. (Atty Docket No.: CAMIP001). That patent application also contains additional material on the mechanisms of CYP enzyme-substrate interactions.

This evidence also shows that the last steps of the CYP catalytic cycle, steps 107 and 108, are not typically the rate-limiting steps in the sense that they are not the slowest steps in the catalytic cycle. They are often the “product-determining” steps, however. While rate-limiting steps are usually thought of as the steps that determine the rate of product formation, if there is an alternate pathway that competes with a fast product formation step, that alternative pathway can unmask the rate of product formation.

Therefore while these last steps in the catalytic cycle do provide useful reaction rate information on substrate metabolism, they do not provide a complete view of substrate metabolism. To determine complete and absolute rates of substrate metabolism, at least the reaction rates of some of the decoupling reactions in the CYP catalytic cycle should be known. In a preferred embodiment, the models of this invention account for at least the peroxide decoupling reactions 105 and 111, and possibly the water decoupling reaction 112.

The water decoupling rate 112 appears to be substrate independent. Its importance in the overall metabolism reaction is based on its relative reaction rate in comparison to step 107. This information finds productive use in metabolism models as described in U.S. patent application Ser. No. 09/613,875, previously incorporated herein by reference.

It appears that the peroxide decoupling steps 110 and 111, unlike the water decoupling reaction 112, is strongly substrate dependent. The models of this invention account for the substrate-dependence of the hydrogen peroxide decoupling reaction. Therefore, the models may make use of certain substrate characteristics (e.g., structural descriptors) to predict the degree to which this decoupling reaction affects the absolute rate of metabolism.

Additional evidence suggests that the decoupling reaction's contribution to the actual reaction rate is a function of the amount of water or hydrophilic structures that a bound molecule presents to a particular region of the reaction site of cytochrome P450s. Thus, the ability of a substrate to exclude water (or hydrophilic structures) from the reaction site can determine the relative contribution of the hydrogen peroxide decoupling reaction.

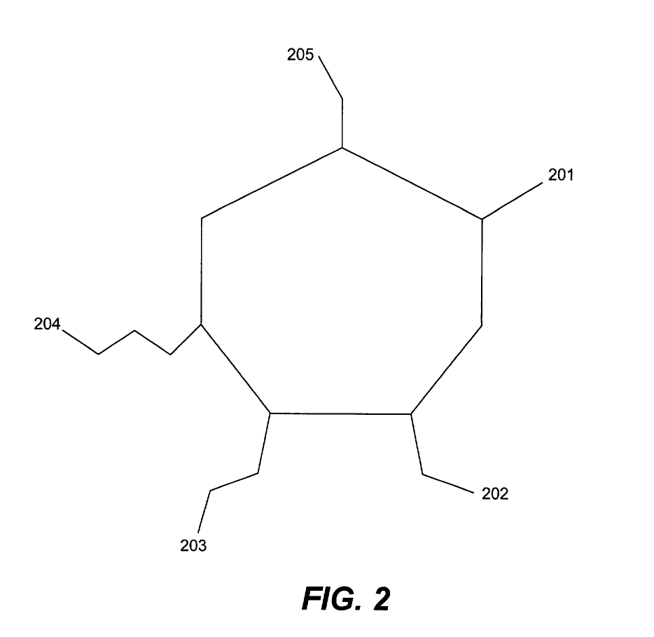

FIG. 2 is a simplified, schematic representation of a substrate molecule with five reactive sites, 201-205, for CYP enzyme metabolism. Each of these sites may serve as the predominate oxidation site for CYP metabolism. Each of these sites may also be subject to one of the decoupling reactions set forth in FIG. 1. In each case, the probability that the site will react during metabolism is a function of the site's intrinsic reactivity in the enzyme's active site and the relative rate of the corresponding decoupling reactions.

One of the most common ADME/PK problems with a drug candidate is that it is metabolized too quickly. In many cases, an ideal drug would be metabolized slowly enough so that it can be administered about once a day. In the current state of the art, if a drug candidate is being metabolized too quickly for daily administration, the designers of the drug will try to redesign it, typically by modifying the most reactive site in a manner that would make it considerably more stable.

However, changing this most reactive site, even by making it extremely stable or even non-reactive, may or may not result in an appreciable decrease in the rate of metabolism of the drug. The result is essentially unpredictable by methods of the current art. For instance, site 203 might be observed to be the most reactive site. A drug designer could then modify it to make more stable or even unreactive in an attempt to decrease the overall metabolic rate of the substrate. In some instances this will be successful, but if the substrate has one or more reactive sites that also have relatively high reactive rates, then these sites will often “take over” the metabolism of the substrate and the overall metabolic rate will remain essentially unchanged.

Therefore, a drug designer would have to go through the time-consuming process of redesigning one site as essentially a shot in the dark, re-testing the ADME/PK properties, and then redesigning that site and/or one or more of the other reactive sites as additional shots in the dark. After conducting this process on most or all of the reactive sites of the drug, the designer might find that it is essentially impossible to achieve the ADME/PK properties that are desired, particularly without reducing, or perhaps destroying, the desired pharmacological properties of the drug. The chances of altering the pharmacological properties of the drug greatly increase as more and more redesigns of the drug are required.

Slowing down the rate of metabolism of a drug candidate is by no means the only ADME/PK property that drug designers try to affect. They also may try to speed up the rate of metabolism of drug. In addition, it is generally preferable that a drug be a substrate for more than one metabolic enzyme, so that chances of dangerous drug interaction, via blocking the primary metabolic pathway, are minimized. The fact that metabolism by multiple enzymes is often desirable, can make the design of the drug even more complicated.

The current invention provides for the modeling of absolute rates of metabolism of a molecule. The complexity of analyzing and modeling a substrate molecule due to multiple reactive sites with relative rate contributions, is discussed in more detail in U.S. patent application Ser. No. 09/613,875, “Relative Rates of P450 Metabolism,” previously incorporated by reference. Thus the current invention can, in one embodiment, be practiced with the referenced patent application to provide a more complete analysis and modeling tool where absolute rates of metabolism are approximated using two or more of the four different reaction pathways for some or all of the P450-substrate complexes. More specifically, this invention accounts for the contribution of the hydrogen peroxide decoupling reaction 111. No currently known technique accounts for the contribution of this decoupling reaction in predicting a given compound's metabolism rate.

2. THE PEROXIDE DECOUPLING PATHWAY

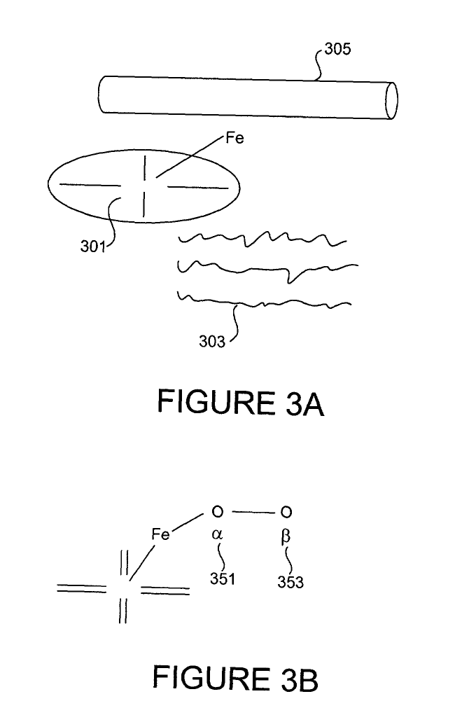

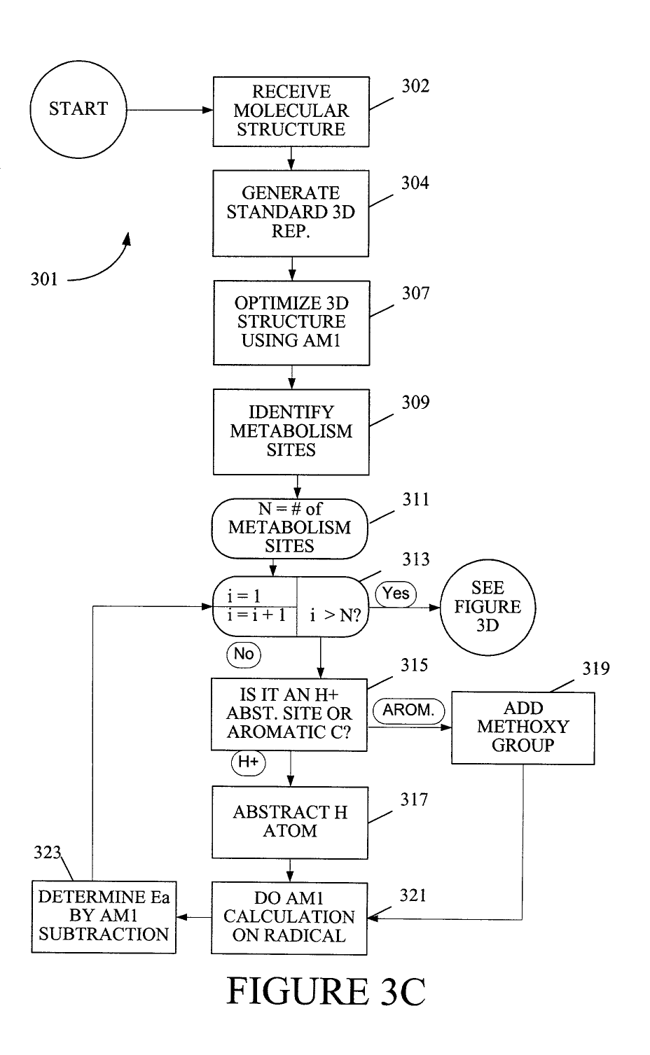

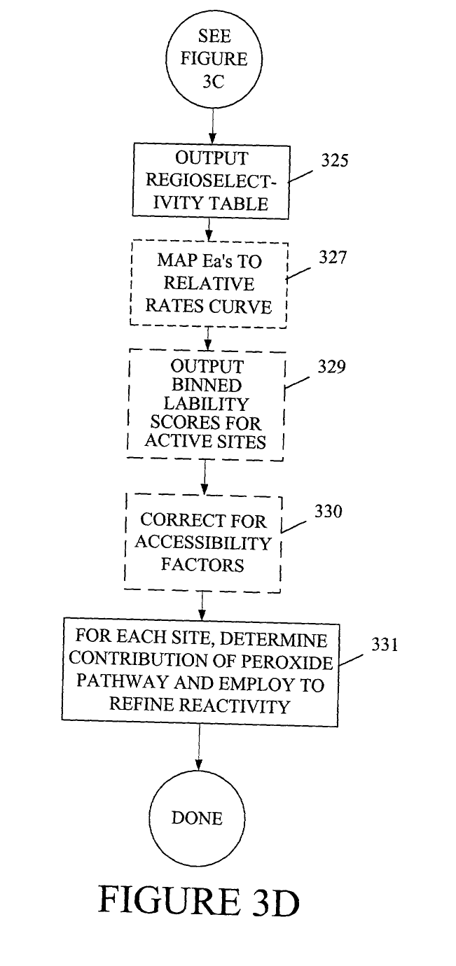

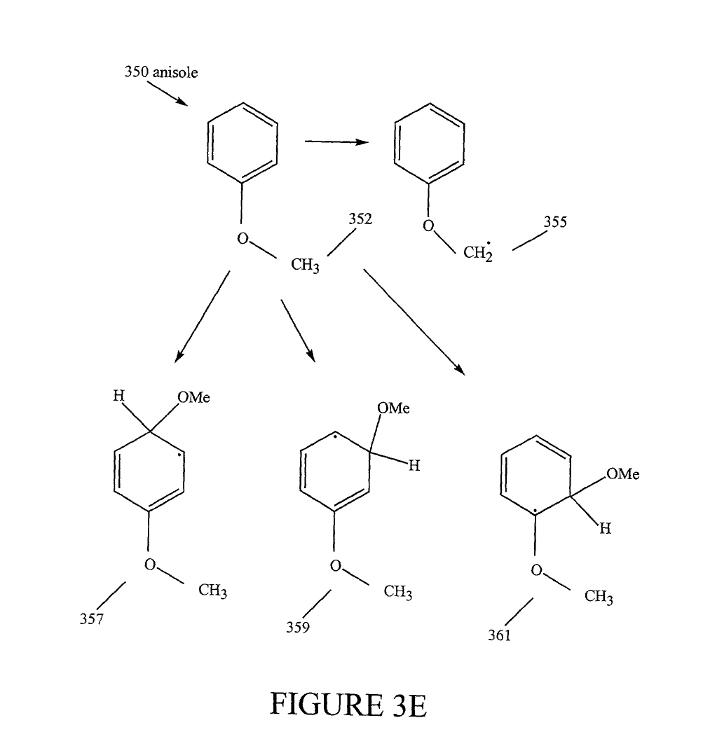

FIG. 3A is a highly simplified schematic illustration of the active site and surrounding region of the CYP enzyme. Crystallography studies show that a heme iron atom 301 sits on top of beta sheets 303 of the enzyme. An “I-helix” section 305, of the enzyme physically overhangs the heme iron atom. In steps 103 through 105 of the catalytic cycle, the heme atom coordinates with molecular oxygen, which is then reduced to the peroxide. FIG. 3B schematically illustrates coordination of the molecular oxygen to the iron atom.

At this point, either of the oxygen atoms in the oxygen molecule can be protonated. Evidence suggests that if the oxygen that is beta to the iron atom 351 is protonated, then the complex undergoes heterolytic cleavage, leaving the single oxygen atom coordinated to the iron atom. This corresponds to step 106 of the catalytic cycle. The substrate may then be oxidized in the metabolic steps 107 and 108 (or undergo the decoupling water branch pathway step 112). Evidence also suggests that if the alpha oxygen 353 is protonated, then the iron-oxygen bond is cleaved from the complex, and the substrate and a peroxide are immediately formed in the non-metabolic step 111.

While not wishing to be bound by theory, it is believed that the structure of the substrate molecule at its site of metabolism dictates whether step 106 or step 111 is favored. Extensive studies have been done on the P450 CAM enzyme, which is a CYP enzyme found in bacteria. Crystallographic studies of P450 CAM and various substrates have shown that when the substrate provides an anhydrous environment around the active site (because of hydrophobic and/or large constituents groups near the site of metabolism that occlude water from the active site), then the heterolytic, metabolic pathway of step 106 is favored. If water is present in certain areas of the active site, then the peroxide decoupling pathway of step 111 is favored. See e.g., Kadkhodayan et al., J. Biol. Chem., Vol. 270, No. 47, pp. 28042-48 (1995), which is incorporated herein by reference for all purposes.

As mentioned, the U.S. patent application Ser. No. 09/613,875, “Relative Rates of Cytochrome P450 Metabolism,” focuses on the relative rate of the last metabolic steps of the CYP catalytic cycle, steps 107 and 108 versus the rate of the decoupling of water branch pathway, step 112. An embodiment of the invention described in that application carries out this comparison for each of the metabolic reactive sites of a substrate molecule, thus generating metabolic characteristics for each reactive site and the molecule as a whole. While this is very useful, it does not always provide an absolute rate of metabolism for a reactive site or a molecule, because of the presence of the oxygen decoupling step 110 and the peroxide decoupling step 111, both of which can contribute to the absolute rate of metabolism.

This invention provides for models that account for the peroxide decoupling step 111 by using information about the substrate and its likelihood of promoting this decoupling reaction. Preferably, the model considers each potential reaction site on a substrate molecule. The relative reactivity of each site may be characterized using the site's intrinsic reactivity (with or without considering the enzyme's specificity) and the possible contribution of the hydrogen peroxide decoupling reaction. Preferably, characteristics of the substrate structure are used to predict an activation energy or rate constant associated with the hydrogen peroxide decoupling reaction.

A specific example of a metabolism model is depicted in FIGS. 3C and 3D. These figures present a flowchart illustrating a high-level process, 300, for predicting site reactivity information using rate constants (or other measures of reactivity) for at least the main substrate metabolism pathway and the hydrogen peroxide decoupling pathway. Process 300 predicts reactivity for arbitrary substrate molecules.

Initially, at operation 302, the molecular structure of a substrate to be characterized is received. The molecular structure can be received as an organic chemistry string of atoms, a two-dimensional structure, a IUPAC standard name, a 3D coordinate map, or as any other commonly used representation. If not already in 3D form, a 3D coordinate map of the molecule is generated, using a geometry program such as Corina or Concord. See 304. The 3D-structure generator Corina is available from Molecular Simulations, Inc., of San Diego, Calif. and Molecular Networks GmbH of Erlange, Germany. Concord is available from Tripos, Inc. of St. Louis, Mo. Corina uses straightforward rules about molecular bond and functional group conformation to generate an approximate geometry 3D structure, which is optimized to a local energy minimum. For instance, if an amide group is encountered, then it will be placed in a planar conformation, as that group normally exists. Concord applies a similar method, but also uses a limited set of molecular mechanical rules involving branch angles, strain and torsion, to achieve its 3D structure.

This approximate 3D-geometry structure is then optimized with a more sophisticated modeling tool that provides an electron distribution for the substrate molecule. See 307. In a specific embodiment, the modeling tool is AM1. AM1 is a semi-empirical quantum-chemical modeling program that optimizes the given 3D structure to that local energy minimum. It calculates electron density distributions from approximate molecular orbitals. It also calculates an enthalpy value for the molecule. AM1 is available as part of the public-domain software package MOPAC, which is available from the Quantum Chemistry Program Exchange, Department of Chemistry, Indiana University, Bloomington, Ind. The MOPAC-2000 version of MOPAC can be obtained from Schrodinger, Inc., of Portland, Oreg.

The process then identifies each reactive site of metabolism on the molecule. See 309. In a specific embodiment, the reactive sites include aliphatic carbons and aromatic carbons. These sites are chosen because CYP enzymes generally oxidize the substrate molecules at these sites. Other reactive sites can be considered in other embodiments, depending on the enzyme and/or class of substrates under consideration. For example, the model might analyze nitrogen atoms, sulfur atoms, allyl carbon atoms, etc. as potential reactive sites. After the set of potential reactive sites has been identified, the process analyzes each reactive site, beginning with operations 311 and 313, where the system sets a variable N equal to the number of reactive sites to be considered (311) and iterates over those sites (311). Iterative loop operation 313 initially sets an index value “i” equal to 1. It then determines whether the current value of i is greater than the value of N. If not, as would be the case on the first iteration, it performs various operations to determine the activation energy (E_{A}) at that site.

In operation 315, the process determines whether the reactive site is an aliphatic carbon or aromatic carbon site (again assuming that only these two types of potential reactive sites are considered). If it is an aliphatic carbon site, the process will remove a hydrogen atom, in silico, from the site. See 317. The process then does a new electron density calculation (using AMI for example) on the molecule to determine its 3D map and enthalpy. See 321. The molecule in this state is an intermediate form of the molecule, which can be used to approximate the transition state through which the molecule will go in the oxidation reaction of step 108. Note that the base molecule's 3D map and enthalpy were calculated at 307. The process then determines the enthalpy difference between the intermediate and base form of the molecule. Assuming that the entropy change of the reaction (ΔS) is close to zero, which is a good assumption for the conditions under which CYP oxidation takes place, the process yields a good approximation of the activation energy value (E_{A}) for the reactive site. Other properties of the radical, such as its ionization potential, can also be used in estimating the E_{A}. A good description of how activation energy may be calculated is described in an article by K. Korzekwa, J. Jones, and J. Gillette, J. Am. Chem. Soc., 112, pp 7042-46 (1990), incorporated herein by reference for all purposes. If the reactive site is an aromatic carbon, then the process will add a methoxy group to the molecule to form the intermediate-radical. See 319. The operations for doing a new electron density (e.g., AM1) calculation, 321, and determining the E_{A}, 323, are the same as they are for proton abstraction sites.

FIG. 3E shows an anisole molecule, 350, which has both an aliphatic and aromatic reaction sites and can be used to illustrate both hydrogen abstraction and methoxy addition. The aliphatic reaction site of the anisole is the terminal methyl group 352. When a hydrogen ion (proton) is abstracted from this group, the intermediate that results has an extra electron on the reactive carbon. See 355. The aromatic ring can react in an ortho, meta or para fashion, with the methoxy group adding to those position as shown in intermediates 357, 359 and 361, respectively. The addition leaves a free electron on the ring.

Note that the activation energy values may be obtained by various techniques. The above-described process employing an enthalpy difference between the base compound and its radical is but one approach to calculating activation energies. Other suitable techniques will be known to those of skill in the art. For example, an approach that maps molecular groups, moieties, or fragments (and their associated environments) to precalculated activation energies may be employed. These other approaches may replace operations 317, 319, 321, and/or 323 of process 300.

When i is greater than N, indicating that all the reactive sites have been analyzed, the process has calculated an activation energy for each reactive site, independently of considerations about the metabolizing enzyme. At this point, the process may output a regioselectivity table or other arrangement of data that indicates the activation energies of each of the reactive sites. See 325. Then, optionally, the activation energies are used to map the reactive sites to a relative rates curve. See 327. This indicates the comparative reaction rate of each site with respect to the water decoupling reaction. If the activation energies are mapped to the reactive sites, they are then binned based upon their relative reaction rates or “lability.” See 329. Details concerning the relative rates curve and its use may be found in U.S. patent application Ser. No. 09/613,875, previously incorporated by reference.

Recognize that the concept of lability is typically specified with reference to a decoupling pathway in the enzyme's catalytic cycle. In the case of the CYP enzymes, as mentioned, the decoupling pathways are illustrated as steps 110, 111, and 112, which are the superoxide, hydrogen peroxide and water decoupling pathways. These decoupling pathways regenerate the unreacted substrate. Substrate reactions with metabolic pathways that compete with, and proceed more rapidly than, these decoupling reactions provide for significantly faster metabolism. The relative rates data of the preferred embodiment specifically applies most directly to the last metabolic steps of the CYP catalytic cycle, steps 106, 107, and 108, as they compare with the water and hydrogen peroxide decoupling pathways.

In order to properly characterize the overall rate of metabolism, the hydrogen peroxide branch pathway must be considered. Thus, at 331, the process refines the absolute reactivity of the molecule. It does this based upon the level to which the hydrogen peroxide decoupling pathway is predicted to affect the reactivity of the molecule. As mentioned, some structural features of the substrate will promote the hydrogen peroxide decoupling reaction. Other structural features will hinder the hydrogen peroxide decoupling reaction. It is believed that when the substrate provides an anhydrous environment around the active site (because of hydrophobic and/or large constituents groups near the site of metabolism that occlude water from the active site), then the metabolic pathway of step 106 is favored. If water can interact with the oxygen molecule, then the peroxide decoupling pathway of step 111 is favored.

Various structural features have been identified as affecting the peroxide decoupling pathway (either promoting or inhibiting it). Application of this invention will identify other structural features that bear on the peroxide decoupling reaction. Regarding reported structural features of interest, Nordblom, G. and Coon, M., Arch. Biochem Biophys, 180:343-347 (1977) shows examples of compound structures that affect the peroxide decoupling pathway. See Table 1. The same is true of Gorsky, L; Koop, D; and Coon, M., J. Biol. Chem, 259: 6812-6817 (1984) (at p. 6814, Tables II and III) and Kadkhodayan, et al., JBiolChem, 270: 28042-28048 (1995) (at p. 28044, Table I showing metabolism variations between camphor and certain substituted camphors). Each of these three references is incorporated herein by reference for all purposes.

Regardless of which structural features actually come into play, they can be characterized and employed with the predictive tools of this invention (at block 331, for example) to predict the relative contribution of the hydrogen peroxide decoupling pathway to the reactivity of the substrate molecule. Typically, this will involve predicting a rate constant or rate expression for the hydrogen peroxide pathway and then using that expression in conjunction with information about the rate of the main substrate metabolism pathway.

To understand how a model may use information about the contribution of the hydrogen peroxide pathway, consider an enzyme having a rate of 0.5 seconds for its catalytic cycle. If the hydrogen peroxide decoupling pathway is predicted to account for 50% of the reaction at branch 111, then 0.25 seconds is the overall rate for substrate oxidation (107) and water formation (112). If the water decoupling branch pathway 112 is predicted to account for 50% of the reaction beyond 106, then the overall rate of substrate oxidation (107) will be 0.125 seconds.

From the above information, the overall reactivity of metabolic characteristics of a given substrate may be obtained by considering, the rates of the main substrate metabolism pathway in conjunction with the branch hydrogen peroxide pathway, and possibly the branch water pathway.

An additional operation that may be required is a steric and/or orientation accessibility correction operation. See 330. As stated earlier, the CYP enzymes, particularly 3A4, are not sterically specific in the way that other enzymes are. However, in certain cases, a reactive site may be deeply buried within the substrate molecule, or the molecule may have a strongly preferred amphoteric orientation, so that the relative rate of the reactive site in metabolism is hindered or accelerated. In such cases, the user may wish to incorporate steric or orientation correction factors. Some of these factors may apply generally to the class of P450 enzymes. Others will be specific to specific P450 enzymes such as 2C9 and 2C6. Systems and methods for incorporating such factors are discussed in U.S. Provisional Patent Application No. 60/127,227, filed Jul. 10, 2000 and U.S. patent application Ser. No. 09/902,470, filed Jul. 9, 2001, both of which were previously incorporated by reference. However, this operation is optional, and in any case the main process of FIGS. 3A and 3B will yield useful information without operation 330.

As mentioned, process 300 considers the contribution of the peroxide decoupling reaction at 331 using information about the substrate. Such information may be provided by a priori analysis of the substrate's structural characteristics, typically with respect to its orientation in the active site. To develop or refine robust models that account for the substrate structure's affect on the decoupling reaction, one may wish to conduct experiments on numerous diverse substrates and measure their relative contributions on the various reaction pathways. From this experimental information, one can identify structural descriptors that govern the relative contributions of the main pathway and the decoupling pathways.

Unfortunately, existing tools for quantifying relevant reactants and products have problems. To develop robust models on a commercially realistic time scale, improved methods are required for obtaining stoichiometric data associated with metabolic reactions.

3. USING STOICHIOMETRY MEASUREMENTS TO DETERMINE THE CONTRIBUTIONS OF THE VARIOUS REACTION PATHWAYS

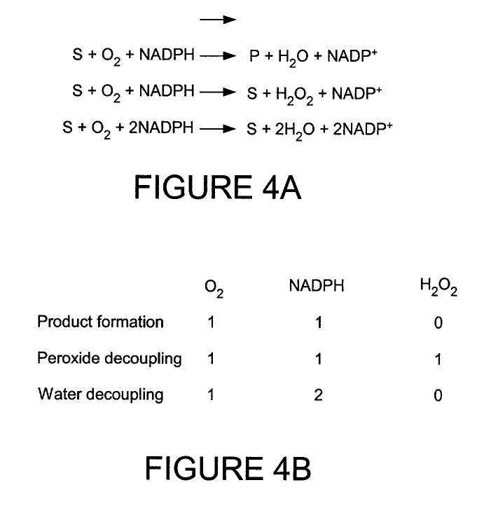

As noted, there are four reaction pathways in the CYP catalytic cycle: the pathway to product, the superoxide decoupling, the peroxide decoupling, and the water decoupling. The catalytic cycle 100 of FIG. 1 illustrates the stoichiometries of these various reactions. The stoichiometry of the superoxide decoupling pathway is given by 2S+2O₂+NADPH=2S+2OO⁻+NADP⁺. The stoichiometry of the product pathway (main pathway) starts with one molecule of substrate (S), one molecule of oxygen and two electrons to yield one molecule of product (P) and one molecule of water. Since NADPH is the electron donor in the catalytic cycle (two electrons per NADPH), we can count the two electrons as an input molecule of NADPH and an output molecule of NADP⁺. This chemical equation, including stoichiometry, is presented as the top equation in FIG. 4A. The chemical equations for the water and hydrogen peroxide pathways, discussed below, are also written out in FIG. 4A.

The stoichiometry of the peroxide branch pathway is one molecule of substrate, one molecule of oxygen and one molecule of NADPH yielding the same one molecule of substrate, one molecule of peroxide and one molecule of NADP⁺. The stoichiometry of the water branch pathway is one molecule of substrate, one molecule of oxygen and two molecules of NADPH yielding the same one molecule of substrate, two molecules of water and two molecules of NADP⁺.

FIG. 4B summarizes the stoichiometric coefficients for oxygen and NAPDH consumption and hydrogen peroxide production for the various reaction pathways. By designing appropriate experiments and determining the relative amounts of reactants consumed and products generated, one can deduce which reaction pathways are present and predominate. In almost any chemical reaction, some sort of stoichiometric deduction can be made given a subset of the ratios of the products formed and/or reactants consumed. For example, a reaction that greatly favors the product-formation pathway will consume molecular oxygen and NADPH at about equivalent rates and will not generate hydrogen peroxide.

For purposes of accurately modeling CYP metabolism, one should determine relative contributions of the product-formation, peroxide-decoupling and water-decoupling pathways. However, it is not necessarily possible or desirable to measure all reactants and/or products of these pathways directly. For instance, measuring the rate of water decoupling could be very difficult if one attempted to measure the water generation directly, since water is ubiquitous in an aqueous medium and would require a controlled isotope-labeling experiment. In addition, the product-formation pathway also produces a molecule of water. Note that for purposes of this discussion, the superoxide branch path is assumed to provide an insignificant contribution.

The species that can be monitored include (listed in degree of difficulty to detect) NADPH, product, oxygen, and hydrogen peroxide. In one preferred embodiment, only (1) oxygen consumption, (2) NADPH consumption, and (3) product formation are measured directly. These three measurements are sufficient to determine the reaction rate for all three pathways in a manner that will be discussed below. Preferably, to facilitate high-throughput analysis, stoichiometric analyses in accordance with this invention typically are carried out in multiple reaction vessels, such as the wells of a 96-well microtitre plate.

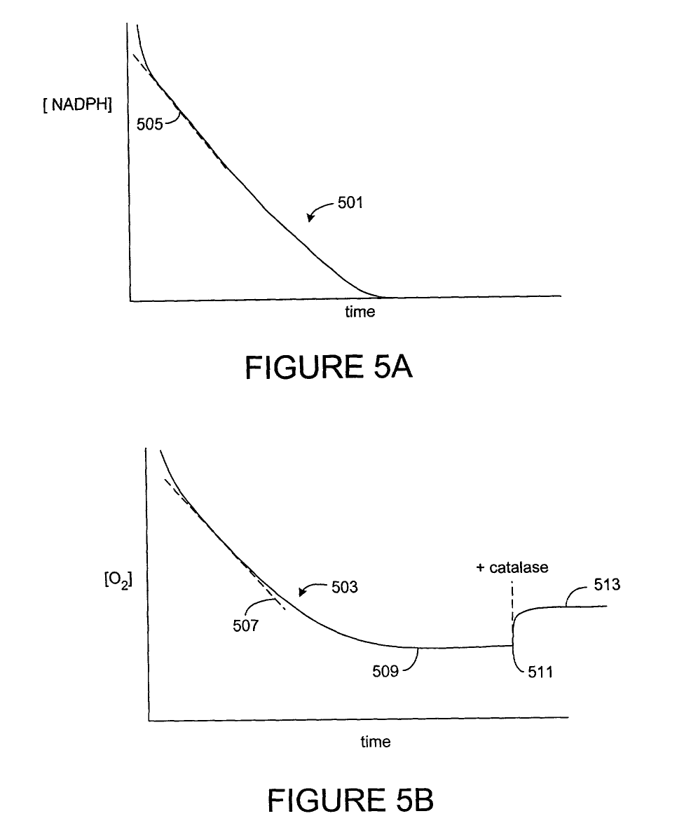

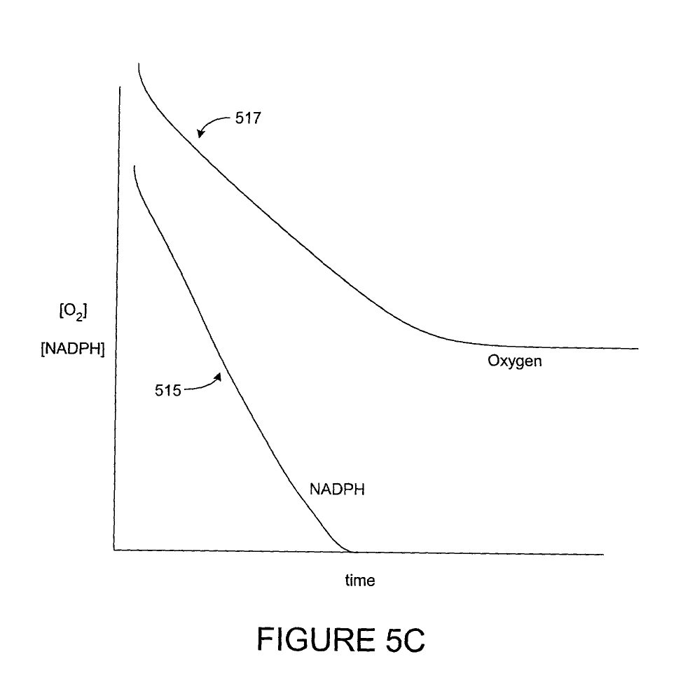

FIGS. 5A AND 5B are graphs depicting the molar concentrations of NADPH and oxygen respectively in experiments including at least substrate molecules, NADPH, reductase, and oxygen as reactants in the presence of one or more CYP enzymes. As shown in FIG. 5A, the concentration of NADPH (indicated by curve 501) decreases rapidly. The slope 505 of the NADPH molar concentration curve 501 indicates how fast the NADPH is being consumed. Similarly, the slope 507 of oxygen molar concentration curve 503 shown in FIG. 5B indicates how fast oxygen is being consumed.

A comparison of the slopes 505 and 507 of the NADPH and oxygen concentration curves 501 and 503 provides some information about which pathway predominates. For example if the ratio of the slopes is 1:1, one can deduce that the pathway is either product formation, hydrogen peroxide formation, or some combination of these pathways. If the ratio is 2:1, one can deduce that the water decoupling reaction predominates. These ratios correspond to the stoichiometric coefficients for the various pathways as indicated in FIG. 4B. If the ratio is between 2:1 and 1:1, one can deduce that the water decoupling reaction is occurring, but does not necessarily predominate.

FIG. 5C further illustrates how the above information may be used. This figure shows curves of NADPH (curve 515) and oxygen (curve 517) molar concentration versus time for a hypothetical experiment. The slope of the NADPH curve is slightly greater than the slope of the oxygen curve. This slope difference indicates that the water decoupling reaction is occurring. If there were no difference in slope, then the water decoupling reaction would not be occurring. The stoichiometric coefficient ratio for NADPH and oxygen is 1 in the product formation pathway and is also 1 in the hydrogen peroxide formation pathway. However, the ratio is 2 for the water generation pathway. Generally, the relative contribution of the water decoupling pathway to the overall metabolism rate can be obtained from the difference between the rate of NADPH consumption and the rate of oxygen consumption: H₂O=NADPH−O₂.

Note that the equilibrium amount of oxygen dissolved in water at standard temperature and pressure is about 220 micromolar. When the concentration of oxygen plateaus at 509 as illustrated in FIG. 5B, the reaction is complete. From this information, one can determine how much oxygen has been consumed.

One can also determine the amount of hydrogen peroxide that has been generated by adding catalase and measuring the increase in oxygen concentration. Catalase is an enzyme that catalyzes decomposition of hydrogen peroxide to generate one molecule of oxygen for every two molecules of hydrogen peroxide.

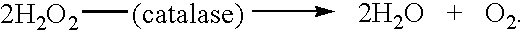

In the example of FIG. 5B, catalase is added at a time 511. In the example shown, the concentration of oxygen thereafter increases to a level 513. This increase in oxygen concentration shows that the peroxide decoupling pathway 111 contributes to the overall reaction. The change in molecular oxygen concentration (given by the difference concentration between levels 513 and 509) corresponds to one-half the concentration of hydrogen peroxide generated during the experiment.

Conventionally, the oxygen concentration is measured by an oxygen electrode. One embodiment involves use of an oxygen electrode probe (with a 2 mm tip, for example), which is immersed and held in the reaction mixture. In this embodiment, the reaction mixture can be in a cuvette so that the oxygen concentration and NADPH concentration can be measured simultaneously. Thus, the conventional techniques for experimentally ascertaining oxygen and hydrogen peroxide stoichiometry cannot be performed rapidly.

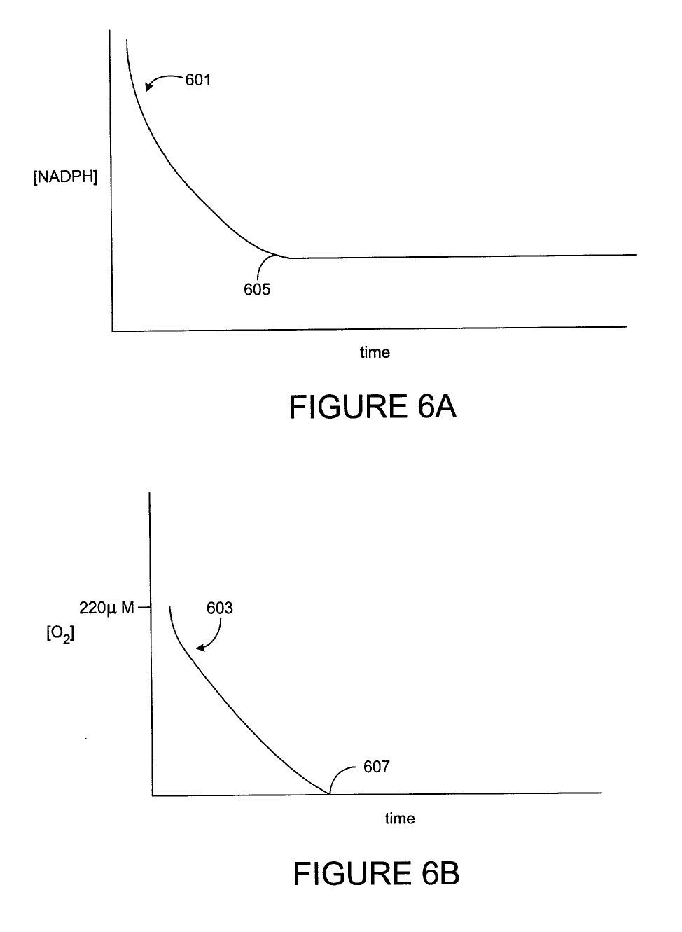

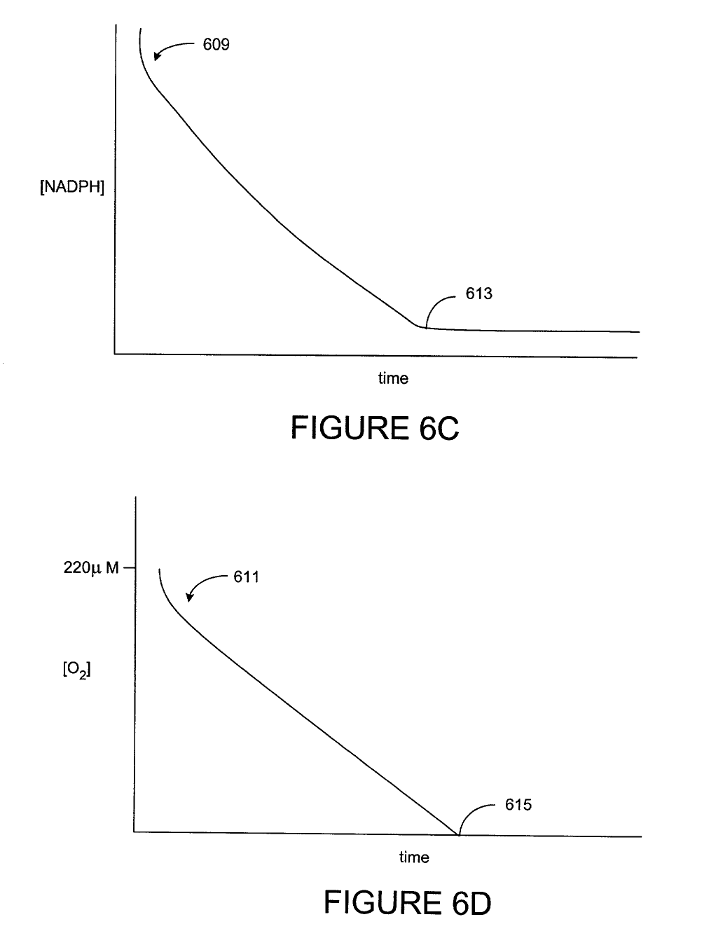

One method for determining oxygen consumption more rapidly involves using a “limiting oxygen” (NADPH plateau) technique as illustrated in FIGS. 6A, 6B, 6C, AND 6D. This approach uses the fact that NADPH concentration can change only as long as the oxygen is present. After that, a needed reactant (oxygen) is absent. (See the balanced chemical equations for the three reaction pathways.)

The experiments producing the data shown in FIGS. 6A-6B employ a known starting concentration of oxygen, substrate, and NADPH. Preferably, the starting concentration of NADPH should be sufficiently high that it will remain non-zero when all oxygen is consumed. Since the stoichiometric ratio of NADPH to oxygen is between 1 and 2, the starting concentration of NADPH should be at least twice that of the oxygen.

When the slope of NADPH concentration curve 601 approaches or equals zero (shown at point 605 in FIG. 6A), one can assume that all oxygen in the reaction vessel has been consumed (indicated at a point 607 in oxygen concentration curve 603 of FIG. 6B). By knowing the amount of oxygen in the vessel at the outset of the experiment (e.g., 220 millimolar), one deduces that that amount of oxygen has been consumed. The NADPH concentration change can be measured simply and directly as described herein.

Because the concentration changes of NADPH and oxygen are known, the molar ratio of NADPH to oxygen consumption is also known. As explained, this indicates whether the water decoupling reaction is occurring; in which case, the molar ratio will be greater than 1:1. In other words, if more NADPH than oxygen is consumed, this indicates that the water decoupling reaction is present. The degree to which this reaction is present depends upon the exact ratio of NADPH to oxygen consumption. If the ratio is near 2:1, then the water decoupling reaction predominates. If the ratio is near 1:1, then the water decoupling reaction is not so important.

The experiment provides the necessary stoichiometric information without directly monitoring the oxygen concentration. Hence, a relatively high throughput technique has been developed for characterizing the enzymatic reaction pathways of particular substrates.

This approach is also useful in monitoring the amount of hydrogen peroxide produced. Hydrogen peroxide decomposes to water and one-half an oxygen molecule in the presence of catalase. So a comparison of catalase + and catalase − reactions will show how much extra oxygen is present by virtue of hydrogen peroxide decomposition.

FIGS. 6C and 6D are analogous to FIGS. 6A and 6B, except that catalase and hydrogen peroxide are presumed present in the experiments used to generate the data of FIGS. 6C and 6D. Thus, in this case, the oxygen concentration (curve 611 of FIG. 6D) decays more slowly because some molecular oxygen is produced by hydrogen peroxide decomposition. Similarly, the NADPH concentration (curve 609 of FIG. 6C) flattens at a later time 613 (corresponding to a point 615 at which the oxygen concentration goes to zero). Comparing the curves in FIGS. 6A (catalase −) AND 6C (catalase +) shows how monitoring NADPH concentration indicates the amount of hydrogen peroxide that has been produced in the reaction. By monitoring the total NADPH consumed for + and − catalase, differences in the NADPH consumed (the difference in NADPH concentration at points 605 and 613) can be directly attributable to the generation of hydrogen peroxide. By comparing the amount of oxygen consumed to the amount of hydrogen peroxide produced, one can deduce the relative contribution of the hydrogen peroxide decoupling pathway. Again, valuable stoichiometric information has been obtained without directly measuring the oxygen concentration.

In order to adjust the dissolved oxygen concentration in the samples to a level where it will be rate limiting, the samples may have to be degassed with argon or nitrogen. Also, enzymatic reactions can be used to decrease the oxygen concentration, especially those that do not produce reactive oxygen species. The firefly luciferin/luciferase system can be used, and has the added benefit of allowing the initial oxygen consumption to be followed via luminometry. The normal oxygen concentration at 37° C. is approximately 200 millimolar. The luciferin concentration in the reaction can be adjusted to consume the desired concentration of oxygen to a predetermined level.

Once the relative contributions of the different pathways have been determined for a substrate molecule, this data is used in constructing a model of substrate metabolism. In addition, the substrate molecule itself, if it is an actual drug candidate, can be redesigned to facilitate or hinder any of the four reaction pathways.

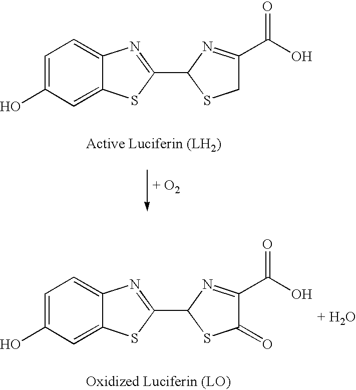

Structural features of the molecule near its reactive sites are used to develop a general model for the absolute rates of CYP metabolism. In just one example, if the peroxide-decoupling pathway of the molecule is found to be unimportant, then the structural groups that are found at and near the reactive sites probably contribute to the occlusion of water from the site. The reaction rates for the pathways are correlated with all the relevant structural features of the molecule until a general, predictive model of absolute rate substrate metabolism is developed (such as the one shown in FIGS. 3C and 3D). Such model is developed and implemented on computer-based systems as described below.

4. CHEMICAL DETECTION TECHNIQUES

To obtain stoichiometry data by the methods of this invention and for producing improved models in accordance with this invention, various chemical techniques may be employed. In each instance, the technique detects a quantity of a particular chemical species. The species of interest include NADPH, substrate (or product), oxygen, and hydrogen peroxide. Typically, the concentrations of such species are measured or monitored after initiating a reaction in a solution or mixture including at least NADPH, oxygen, substrate, a relevant metabolizing enzyme, and, if appropriate, catalase.

The concentration of oxygen in a sample can be measured by at least two methods, (1) using platinum electrodes (also referred to herein as “oxygen electrodes”) and (2) using luminescent ruthenium complexes. As mentioned, electrochemical oxygen measurements provide data very slowly. This is because the platinum electrodes used to monitor oxygen consumption take a long time to reach a stable state.

In one embodiment, the ruthenium luminescent compound used is dichlorotris (1,10-phenanthroline) ruthenium(II), hydrate (DP-Ru), which can be modified to become water-soluble. DP-Ru fluoresces at 600 nanometers when illuminated with blue light (470 nanometers), with a luminescent lifetime that is a function of the partial pressure of oxygen.

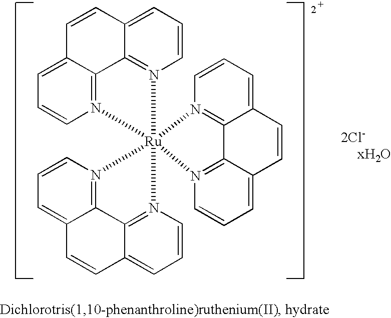

The DP-Ru can be present in solution, bound to a linker at the bottom of the reaction vessel, or encased in a solid polymer matrix such as polyacrylamide. Time-resolved fluorometry is typically used to determine the concentration of oxygen. If the reactions are being carried on a microtitre plate, then each well needs to be separately analyzed for luminescence. DP-Ru can be made soluble by converting it to a sulfonate. This process is described in Castellano, F. N. and Lakowicz, J. R., Photochemistry and Photobiology 67(2): 179-183 (1998), which is incorporated herein by reference for all purposes.

NADPH consumption is typically measured by ultraviolet (UV) absorption. NADPH absorbs at 340 nanometers, and the absorption is proportional to concentration. Typically, UV absorption is monitored by passing UV radiation through a cuvette containing the solution of interest. In a preferred embodiment, the UV absorption is measured in microtiter well or other small reaction vessel.

Substrate and product concentrations can be detected/monitored by any suitable technique. In a preferred embodiment, the substrate or product concentration is measured by a chromatography and/or spectrographic technique that is well known in the art such as HPLC or LCMS.

Hydrogen peroxide concentration can be measured by the use of N-acetyl-3, 7-dihydroxyphenoxazine (Amplex Red). Hydrogen peroxide stoichiometrically converts Amplex Red into the fluorescent compound, Resorufin. Resorufin has an excitation maximum of 563 nanometers and an emission maximum of 587 nanometers. The published limit of sensitivity of Amplex Red is approximately 5 picomoles of hydrogen peroxide.

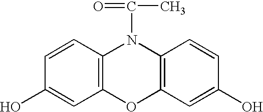

Amplex Red (N-acetyl-3, 7-dihydroxyphenoxazine)

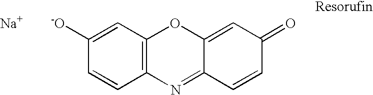

As indicated above, hydrogen peroxide can be indirectly measured by catalyzing its reaction to produce oxygen. The oxygen concentration can be measured directly by one of the above techniques or indirectly by monitoring the NADPH concentration when the initial concentration of oxygen is known. For example, the NADPH or oxygen concentration can be monitored in separate reaction vessels, one of which includes catalase and the other of which does not contain catalase. As indicated above in the discussion of FIGS. 6A-6D, one can compare the levels of NADPH or oxygen in the catalase+ and catalase− experiments to determine how much hydrogen peroxide has been generated. The molar excess of oxygen in the catalase+ experiments corresponds to one-half the molar amount of hydrogen peroxide generated during the reaction.

Alternatively, as shown in FIG. 5B, one can run the substrate reaction to completion, and then add catalase. The molar amount of hydrogen peroxide that was produced by the reaction is equal to two times the molar amount oxygen produced by the catalase catalyzed decomposition.

5. APPARATUS FOR MEASURING REACTANT AND PRODUCT CONCENTRATIONS

In certain specific embodiments in which oxygen is a reaction-limiting reactant, the initial concentration of oxygen in the reaction system must be carefully controlled. Further, no additional oxygen must enter the system from the ambient. As mentioned, the total oxygen concentration can be measured or otherwise determined before the reaction, and this will equal the amount consumed (the concentration of oxygen will go to zero as the reaction completes). Thus, the reaction system should be closed to the ingress of oxygen before and during the reaction and measurements of the reactants and products. Further, the reaction system should not include a significant quantity of oxygen that can serve as a reservoir to provide more dissolved oxygen to the reaction system during experiments.

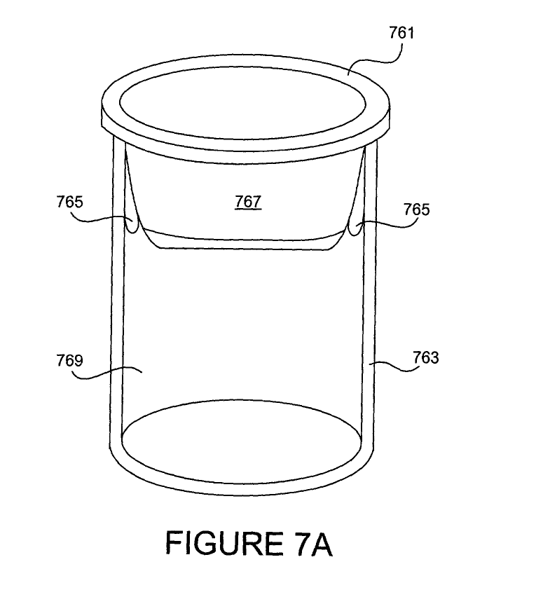

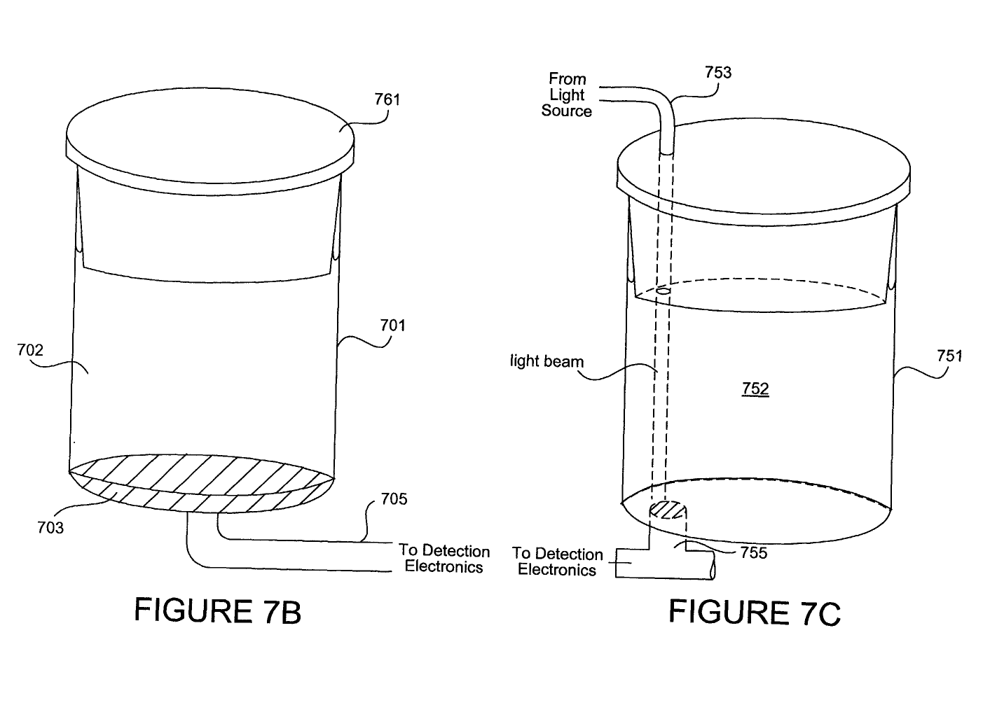

In one embodiment, small reaction vessels having caps to prevent ingress of oxygen are employed. An example of this cap is illustrated schematically in FIG. 7A. As shown, a reaction well 763 (e.g., a well of a 96-well microtiter plate) having a volume of between about 0.2 and 0.5 milliliters is provided with a cap 761 that fits into the top of well 763. Even with the cap 761, the reaction well 763 will typically have some airspace above it. In a preferred embodiment, the cap and vessel are designed to create a steeply curved meniscus 765 at the surface of a reaction solution 769. Specifically, cap 763 includes a plug portion 767 that extends down into well 763, approximately ¼ of the volume of the well, to contact a reaction solution 769 so that the solution wets cap 761 to form meniscus 765. As shown, the meniscus forms in the region between the edge of plug portion 767 and the vertical circumferential wall of well 763. Oxygen diffusion into the solution is substantially prevented or reduced to an insignificant level by the meniscus.

In a preferred embodiment, the sample may be initially degassed with a non-reactive gas such as argon or nitrogen. In another embodiment, the excess oxygen is consumed by using the firefly luciferin/luciferase system, as described above.

Sometimes fluorescent or luminescent species are employed to monitor the concentration of a reactant or product. As mentioned, DP-Ru can be present in solution, bound to a linker at the bottom of the reaction vessel, or encased in a solid polymer matrix such as polyacrylamide. Time-resolved fluorometry is typically used to determine the concentration of oxygen. If the reactions are being carried on a microtitre plate, then each well needs to be separately analyzed for luminescence. In a preferred embodiment, fiber optic probes are used, with one probe for each well. FIG. 7B illustrates schematically a microtitre well or other small volume reaction vessel 701 holding a defined quantity of reaction solution 702. In this example, DP-Ru (or other fluorescent or luminescent species) is embedded in a polymer matrix 703 (e.g., polyacrylamide) and affixed to the base of vessel 701. In alternative embodiments, the fluorescent or luminescent species is dissolved or dispersed in the reaction solution 702 or affixed elsewhere in vessel 701. A fiber optic probe 705 is attached to vessel 701 in a manner that allows detection of light intensity at an emission wavelength of the fluorescent or luminescent species. If a fluorescent species is employed, then a source of excitation radiation, not shown, must be provided. In one example, excitation radiation of the required wavelength is provided via a lamp or other source that illuminates the reaction vessel from above or from the side.

Sometimes reactants or products are monitored by measuring the optical absorption of a reaction solution. For example, NADPH consumption may be measured by UV absorption. NADPH absorbs at 340 nanometers, and the absorption is proportional to concentration. In a preferred embodiment, the UV absorption is measured in microtiter well or other small reaction vessel. FIG. 7C schematically illustrates a microtitre well or other small reaction vessel 751 holding a defined quantity of reaction solution 752. UV radiation from a UV source 753 located at one end of well 751 passes through solution 752 and is partially absorbed by NADPH. A UV detector 755 located at the other end of well 751 detects the intensity of UV radiation passing through solution 752. The intensity of the radiation detected by detector 755 corresponds to the concentration of NADPH in the defined volume of solution 752. Using this apparatus, one can carefully monitor the progress of an enzymatic reaction via NADPH consumption.

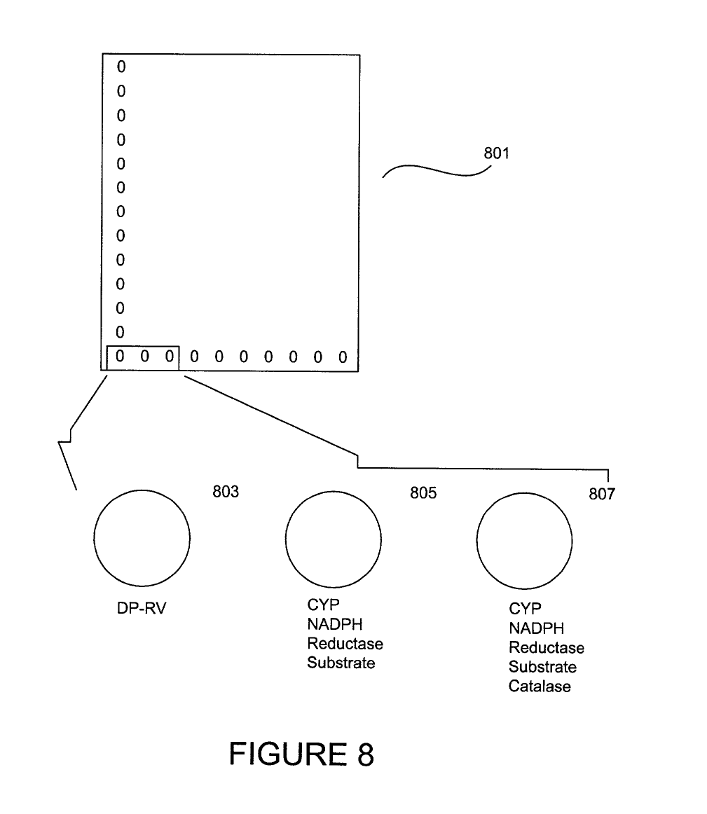

In a specific embodiment, three separate wells from a microtitre plate are used to test each substrate molecule. A plate 801 having such combination of wells is illustrated schematically in FIG. 8. The oxygen is reduced to a reaction-limiting level (e.g., at most about 50 μM) in each of the wells. The first well 803 contains the Ruthenium complex, for example, and is employed to accurately determine the oxygen concentration. The other two wells 805 and 807 contain NADPH, the substrate molecule and reductase (reductase is an enzyme that hands off electron from NADPH to CYP). One of these wells (well 807) contains catalase, the other (well 805) does not. CYP is then added to both of these wells to start the reaction. NADPH consumption is measured throughout the reaction and to its completion, as a function of UV absorption, which yields curves similar to those illustrated in FIGS. 6A and 6C. Specifically, the curve obtained from well 805 might correspond to that shown in FIG. 6A and the curve obtained from well 807 might correspond to that shown in FIG. 6C. The slope of the curves represents the rate of NADPH consumption, and indirectly, the rate of oxygen consumption/generation.

The difference between oxygen consumption and NADPH consumption (for well 805 without catalase) indicates the rate of the water-decoupling pathway. The difference between the NADPH consumption for well 805 without catalase and well 807 with catalase indicates the rate of the peroxide-decoupling pathway. Thus, the embodiment of FIG. 8 allows researchers to rapidly ascertain the relative contributions of the water and hydrogen peroxide decoupling pathways in the metabolism a particular substrate.

6 . HARDWARE AND SOFTWARE IMPLEMENTATION OF THE INVENTION

FIGS. 9A and 9B illustrate a computer system 900 suitable for implementing embodiments of the present invention. Preferably, the apparatus is used to run models, such as process 300, that predict metabolic properties of substrates in accordance with this invention.

FIG. 9A shows one possible physical form of the computer system. Of course, the computer system may have many physical forms ranging from an integrated circuit, a printed circuit board and a small handheld device up to a huge super computer depending on the processing requirements of the embodiment. Computer system 900 includes a monitor 902, a display 904, a housing 906, a disk drive 908, a keyboard 910 and a mouse 912. Disk 914 is a computer-readable medium used to transfer data to and from computer system 900.

FIG. 9B is an example of a block diagram for computer system 900. Attached to system bus 920 are a wide variety of subsystems. Processor(s) 922 (also referred to as central processing units, or CPUs) are coupled to storage devices including memory 924. Memory 924 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable of the computer-readable media described below. A fixed disk 926 is also coupled bi-directionally to CPU 922; it provides additional data storage capacity and may also include any of the computer-readable media described below. Fixed disk 926 may be used to store programs, data and the like and is typically a secondary storage medium (such as a hard disk) that is slower than primary storage. It will be appreciated that the information retained within fixed disk 926, may, in appropriate cases, be incorporated in standard fashion as virtual memory in memory 924. Removable disk 914 may take the form of any of the computer-readable media described below.

CPU 922 is also coupled to a variety of input/output devices such as display 904, keyboard 910, mouse 912 and speakers 930. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 922 optionally may be coupled to another computer or telecommunications network using network interface 940. With such a network interface, it is contemplated that the CPU might receive information from the network, or might output information to the network in the course of performing the above-described method operations. Furthermore, method embodiments of the present invention may execute solely upon CPU 922 or may execute over a network such as the Internet in conjunction with a remote CPU that shares a portion of the processing.

In addition, embodiments of the present invention further relate to computer storage products with a computer-readable medium that have computer code thereon for performing various computer-implemented operations such as running or executing machine readable instructions for performing the metabolism rate models of this invention. The media and computer code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs), ROM and RAM devices, and signal transmission media for delivering computer-readable instructions, such as local area networks, wide area networks, and the Internet. Examples of computer code include machine code, such as produced by a compiler, and files containing higher level code that are executed by a computer using an interpreter. The invention also pertains to carrier waves and transport media on which the data and instructions of this invention may be transmitted.

FIG. 10 is a schematic illustration of an Internet-based embodiment of the current invention. See 1000. According to a specific embodiment, a client 1002, at a drug discovery site, for example, sends data 1008 identifying organic molecules 1008 to a processing server, 1006 via the Internet 1004. The organic molecules are simply the molecules that the client wishes to have analyzed by the current invention. At the processing server 1006, the molecules of interest are analyzed by a model 1012, which predicts overall metabolism rates or hydrogen peroxide decoupling contribution, for example. The processing server may also redesign compounds to improve their metabolism properties.

After the analysis, the predicted metabolism information 1010 (and any other appropriate information) are sent via the Internet 1004 back to the client 1002. The computer system illustrated in FIGS. 9A and 9B is suitable both for the client 1002 and the processing server 1006. In a specific embodiment, standard transmission protocols such as TCP/IP (transmission control protocol/internet protocol) are used to communicate between the client 1002 and processing server 1006. Security measures such as SSL (secure socket layer), VPN (virtual private network) and encryption methods (e.g., public key encryption) can also be used.

Although various details have been omitted for brevity's sake, certain design alternatives may be implemented. Therefore, the present examples are to be considered as illustrative and not restrictive, and the invention is not to be limited to the details given herein, but may be modified within the scope of the appended claims.

What is claimed is:

1. A method, implemented on a computer, of predicting the relative reaction velocities of (i) a first reaction pathway in which a substrate reacts in the presence of a cytochrome P450 enzyme to form a product and (ii) a second pathway in which hydrogen peroxide is produced from a complex of the cytochrome P450 enzyme and the substrate in a manner that regenerates the substrate without producing the product, the method comprising:
(a) analyzing the molecular structure of the substrate to identify a structural feature that affects the rate at which hydrogen peroxide is formed in the active site of the cytochrome P450 enzyme; and
(b) predicting the relative contributions of the first and second reaction pathways based on the identity of the structural feature, to thereby predict the relative reaction velocities.

2. The method of claim 1, wherein the structural feature at least partially excludes at least one of water and hydrophilic components from a defined region of the cytochrome P450 enzyme's reactive site.

3. The method of claim 1, further comprising predicting the relative contribution of a water decoupling pathway for each reactive site.

4. A computer program product comprising a machine readable medium on which is provided instructions for predicting the relative reaction velocities of (i) a first reaction pathway in which a substrate reacts in the presence of a cytochrome P450 enzyme to form a product and (ii) a second pathway in which hydrogen peroxide is produced from a complex of the cytochrome P450 enzyme and the substrate in a manner that regenerates the substrate without producing the product, the instructions specifying a method comprising:
(a) analyzing the molecular structure of the substrate to identify a structural feature that affects the rate at which hydrogen peroxide is formed in the active site of the cytochrome P450 enzyme; and
(b) predicting the relative contributions of the first and second reaction pathways based on the identity of the structural feature, to thereby predict the relative reaction velocities.

5. The computer program product of claim 4, wherein the structural feature at least partially excludes at least one of water and hydrophilic components from a defined region of the cytochrome P450 enzyme's reactive site.

6. The computer program product of claim 4, wherein the method further comprises predicting the relative contribution of a water decoupling pathway for each reactive site.

7. A method of developing a model for predicting the rate at which a substrate is metabolized by a cytochrome P450 enzyme, the method comprising:
(a) using stoichiometric data to characterize
(i) the reaction rate of a first reaction pathway in which the substrate reacts in the presence of the cytochrome P450 enzyme to form a product
(ii) the reaction rate of a second pathway in which hydrogen peroxide is produced from a complex of the cytochrome P450 enzyme and the substrate in a manner that regenerates the substrate without producing the product, and
(iii) the reaction rate of a third pathway in which water is produced from a second complex of the cytochrome P450 enzyme and the substrate in a manner that regenerates the substrate without producing the product; and
(b) specifying one or more reaction rate expressions or rate constants derived using the stoichiometric data for the first, second, and third pathways, to thereby allow a test substrate to be evaluated by said reaction rate expressions or rate constants.

8. The method of claim 7, wherein using stoichiometric data to characterize the reaction pathways comprises using stoichiometric data for multiple substrates.

9. The method of claim 7, wherein the stoichiometric data is obtained from experiments that measure the concentration of NADPH.

10. The method of claim 9, wherein the experiments also measure the concentration of hydrogen peroxide.

11. The method of claim 9, wherein the experiments also measure the concentration of at least one of oxygen and a product of the metabolism.

12. For a catalytic cycle involving an enzyme and a substrate, which catalytic cycle has (a) a first reaction pathway in which a substrate reacts in the presence of the enzyme to form a product and (b) a second pathway in which water is produced from a complex of the enzyme and the substrate in a manner that regenerates the substrate without producing the product, a method of determining the relative contribution of the second pathway, the method comprising:
determining a change in concentration of NADPH during a reaction involving at least the following reactants: NADPH, the enzyme, and the substrate;
determining a change in concentration of oxygen during the reaction; and
estimating the relative contribution of the second pathway based upon the relative values of the change in concentration of NADPH and the change in concentration of oxygen.

13. The method of claim 12, wherein the change in oxygen concentration is determined without directly measuring oxygen concentration during the reaction.

14. The method of claim 12, wherein estimating the relative contribution of the second pathway comprises determining the excess amount of the change in concentration of NADPH over the change in concentration of oxygen.

15. For a catalytic cycle involving an enzyme and a substrate, which catalytic cycle has (a) a first reaction pathway in which a substrate reacts in the presence of the enzyme to form a product and (b) a second pathway in which hydrogen peroxide is produced from a complex of the enzyme and the substrate in a manner that regenerates the substrate without producing the product, a method of determining the relative contribution of the second pathway, the method comprising:
conducting a first reaction involving at least the following reactants: NADPH, the enzyme, and the substrate;
determining a change in concentration of oxygen during the first reaction;
conducting a second reaction involving at least the following reactants: NADPH, the enzyme, the substrate, and catalase;
determining a change in concentration of oxygen during the second reaction;
comparing the change in oxygen concentrations in the first and second reactions to determine the amount of hydrogen peroxide produced via the catalytic cycle.

16. The method of claim 15, wherein the first and second reactions are conducted simultaneously.

17. The method of claim 15, wherein the change in oxygen concentration is determined by measuring the change in NADPH concentration.

18. The method of claim 15, wherein comparing the change in oxygen concentrations in the first and second reactions comprises determining the excess amount of the change in concentration of oxygen in the second reaction over the change in concentration of oxygen in the first reaction.

19. A method generating a model of substrate metabolism using the rate or amount of water formation for a substrate molecule in a cytochrome P450 catalyzed reaction, the method comprising:
(a) measuring the rate or amount of consumption of oxygen;
(b) measuring the rate or amount of consumption of NADPH;
(c) comparing the rates or amounts of consumption of oxygen and NADPH to determine the rate or amount of water formation from a water decoupling branch pathway for the substrate molecule in the cytochrome P450 catalyzed reaction, and
(d) generating a general model of substrate metabolism based on molecular structure.

20. The method of claim 19, further comprising calculating a reaction rate for the water-decoupling pathway of the substrate molecule.

21. The method of claim 19, wherein the concentration of oxygen is reduced to a reaction-limiting amount prior to the cytochrome P450 catalyzed reaction.

22. The method of claim 19 wherein (a) (b) and (c) are repeated for at least two substrate molecules.

23. The method of claim 19 wherein (a) (b) (and (c) are repeated for a class of substrate molecules.

24. The method of claim 19 wherein the rate or amount of oxygen consumption is measured using a ruthenium complex matrix.

25. The method of claim 20 wherein the reaction rate is calculated based on the difference between oxygen and NADPH consumption.

26. A method for calculating the rate or amount of consumption of peroxide formation for a substrate molecule in a cytochrome P450 catalyzed reaction, the method comprising:
(a) measuring the rate or amount of consumption of oxygen;
(b) measuring the rate or amount of consumption of NADPH;
(c) adding catalase to the reaction; and
(d) measuring the rate or amount of formation of oxygen.

27. The method of claim 26 further comprising calculating a reaction rate for the peroxide-decoupling pathway of the substrate molecule.

28. The method of claim 26 wherein the amount of oxygen is reduced to a reaction-limiting amount.

29. The method of claim 26 wherein (a) (b) (c) and (d) are repeated for at least two substrate molecules.

30. The method of claim 26 wherein (a) (b) (c) and (d) are repeated for a class of substrate molecules.

31. The method of claim 26 further comprising generating a general model of substrate metabolism based on molecular structure.

32. The method of claim 26 wherein the rate or amount of oxygen consumption is measured using a ruthenium complex matrix.

33. A method for modeling reaction rates of cytochrome P450 metabolism for substrate molecules, the method comprising:
(a) receiving or generating a peroxide-decoupling rate and a molecular structure for a substrate molecule; and
(b) receiving or generating a water-decoupling rate and a molecular structure for a substrate molecule.

34. The method of claim 33 wherein (a) and (b) are repeated for at least two substrate molecules.

35. The method of claim 33 wherein (a) and (b) are repeated for a class of substrate molecules.

36. The method of claim 33 further comprising receiving a product-formation rate and a molecular structure for a substrate molecule.

37. A method for modeling absolute rates of cytochrome P450 metabolism for substrate molecules, the method comprising:
creating a computational model for peroxide-decoupling rate based on substrate molecule structures; and
creating a computational model for water-decoupling rate based on substrate molecule structures.

38. The method of claim 37 further comprising creating a computational model for product-formation pathway rate based on substrate molecule structures.

39. A method of predicting the relative reaction velocities of (a) a first reaction pathway in which a substrate reacts in the presence of a cytochrome P450 and (b) a second pathway the method comprising:
analyzing the molecular structure of the substrate to determine if it possesses a particular structural feature; and
predicting whether the first reaction pathway is preferred when the substrate possesses said structural feature.

40. The method of claim 39 wherein the first reaction pathway is a product-formation pathway.

41. The method of claim 39 wherein the first reaction pathway is a peroxide-decoupling pathway.

42. The method of claim 39 wherein the particular structural feature is one that excludes water from the substrate reactive site.

43. The method of claim 39 wherein the particular structural feature is one that does not exclude water from the substrate reactive site.

44. The method of claim 39 wherein the particular structural feature is hydrophobic.

45. The method of claim 39 wherein the particular structural feature is hydrophilic.

46. A computer program product comprising a machine readable medium on which is provided instructions for executing a method of predicting the relative reaction velocities of (a) a first reaction pathway in which a substrate reacts in the presence of a cytochrome P450 and (b) a second pathway the method comprising:
analyzing the molecular structure of the substrate to determine if it possesses a particular structural feature; and
predicting whether the first reaction pathway is preferred when the substrate possesses said structural feature.

47. The computer program product of claim 46 wherein the first reaction pathway is a product-formation pathway.

48. The computer program product of claim 46 wherein the first reaction pathway is a peroxide-decoupling pathway.
